(19) United States
(12) Patent Application Publication
(10) Pub. No.: US 2025/0295811 A1
(43) Pub. Date: Sep. 25, 2025

(54) USE OF RS1 GENE IN PREPARING THERAPEUTIC AGENT FOR XLRS, AND THERAPEUTIC AGENT

(71) Applicant: WUHAN ZHONGMOU BIOTECHNOLOGY CO., LTD., Wuhan, Hubei (CN)

(72) Inventor: Yin SHEN, Hubei (CN)

(73) Assignee: WUHAN ZHONGMOU BIOTECHNOLOGY CO., LTD., Wuhan, Hubei (CN)

(21) Appl. No.: 18/860,939

(22) PCT Filed: Apr. 18, 2023
(86) PCT No.: PCT/CN2023/088958

§ 371 Date: Oct. 28, 2024

(30) Foreign Application Priority Data
Apr. 28, 2022 (CN) 202210460778.5

(51) Int. Cl. A61K 48/00; C07K 14/47; C12N 15/86

(52) U.S. Cl. CPC A61K 48/0058; C07K 14/47; C12N 15/86; C12N 2750/14143; C12N 2830/15

(57) ABSTRACT

Use of the RS1 gene in preparing a therapeutic agent for X-linked juvenile retinoschisis is provided. The RS1 gene in an RS1 gene expression cassette is subjected to codon optimization, and the nucleotide sequence of said gene is as shown in SEQ ID NO: 6. In an in-vitro expression experiment, the mRNA level of the RS1 gene expression cassette after codon optimization was about five times that of the original RS1 gene expression cassette, and the protein level was about seven times that of the original.

### TECHNICAL FIELD

The present application relates to the field of ophthalmic disease treatment and molecular medicine, specifically to use of RS1 gene in preparing a therapeutic agent for X-linked retinoschisis.

### BACKGROUND ART

X-linked retinoschisis (XLRS) is a rare retinal degeneration disease caused by RS1 gene mutation, and its incidence rate is about 1:25000 to 1:5000. This disease is inherited in a recessive manner on the X chromosome, with male onset being more common and females often being asymptomatic carriers (George N D, et al. X linked retinoschisis. Br J Ophthalmol, 1995, 79 (7): 697-702. George N D, et al. Clinical features in affected males with X-linked retinoschisis. Arch Ophthalmol, 1996, 114 (3): 274-80). Retinoschisis often occurs between the retinal nerve fiber layer and the ganglion cell layer, which can lead to severe visual impairment, macular foveal split, and disproportionate decrease of b-wave relative to a-wave in electroretinogram (Molday R S, et al. X-linked juvenile retinoschisis: clinical diagnosis, genetic analysis, and molecular mechanisms. Prog Retin Eye Res, 2012, 31 (3): 195-212). At present, clinical practice mainly focuses on observation and treatment of complications, and there is no effective treatment method yet.

With more and more treatments using adeno-associated virus (AAV) as a delivery vector, gene therapy as a fundamental treatment method provides new possibilities for the treatment of XLRS. However, at present, there is no sufficient effective delivery vector constructed and screened to deliver the normal RS1 gene into the body for gene therapy on XLRS.

### SUMMARY

To solve the above problems, the present application provides use of RS1 gene in preparing a therapeutic agent for X-linked retinoschisis.

Preferably, the sequence of the RS1 gene is shown as SEQ ID NO: 6.

The present application also provides a therapeutic agent for X-linked retinoschisis, comprising an RS1 gene expression vector, wherein the RS1 gene expression vector comprises an RS1 gene expression cassette.

In a specific embodiment, a sequence of RS1 gene in the RS1 gene expression cassette is a codon optimized sequence, which is shown as SEQ ID NO: 6. In an in-vitro expression experiment, the mRNA level and the protein level of the RS1 gene expression cassette after codon optimization are about 5 times and 7 times those of the original RS1 gene expression cassette, respectively.

In a specific embodiment, a promoter that controls the expression of the RS1 gene is a CMV promoter.

In a specific embodiment, the RS1 gene expression cassette further comprises a non-coding regulatory sequence, which comprises one or more of an Intron sequence, a Kozak sequence, a 5′UTR sequence, a WPRE sequence, and a HGHpA sequence.

In a preferred embodiment, the Intron sequence is shown as SEQ ID NO: 4, the Kozak sequence is shown as SEQ ID NO: 5, the WPRE sequence is shown as SEQ ID NO: 7, the HGHpA sequence is shown as SEQ ID NO: 3, and the 5′UTR sequence is selected from the group consisting of SEQ ID NO: 8 and SEQ ID NO: 9, or is composed of SEQ ID NO: 8 and SEQ ID NO: 9 connected in series. In an embodiment with concatenation, the 5′UTR sequence is shown as SEQ ID NO: 10. By codon optimization and design of the non-coding regulatory sequence in the construction of the expression vector, the expression efficiency of the RS1 gene expression cassette is improved, so that the mRNA level and the protein level thereof increase to 12-16 times those of the existing expression vector.

In a preferred embodiment, the RS1 gene expression cassette is CMV-Indon-5′UTR-Kozak-hRS1-WPRE-HGHpA.

In a specific embodiment, the expression vector is an adeno-associated virus expression vector, which is packaged in an adeno-associated virus particle.

Preferably, the adeno-associated virus is AAV2/8.

The present application provides use of the RS1 gene or the therapeutic agent comprising an RS1 gene expression vector for treating X-linked retinoschisis.

The present application also provides use of the therapeutic agent comprising an RS1 gene expression vector in the preparation of a medicament for treating X-linked retinoschisis.

The present application also provides a method for treating X-linked retinoschisis, comprising administering the therapeutic agent comprising the RS1 gene expression vector to a subject in need thereof.

In the present application, a therapeutic agent for the effective treatment of X-linked retinoschisis is obtained by constructing an RS1 gene expression vector with highly efficient expression. After the therapeutic agent is administrated into the vitreous cavity by injection, the expression of RS1 protein can significantly be increased, thereby achieving the goal of treating X-linked retinoschisis. The therapeutic agent has the potential to become a candidate drug for treating X-linked retinoschisis.

### BRIEF DESCRIPTION OF THE DRAWINGS

FIG. 1 shows the plasmid profile of expression vector 1.

FIG. 2 shows the plasmid profile of expression vector 2.

FIG. 3 shows the plasmid profile of expression vector 3.

FIG. 4 shows the plasmid profile of expression vector 4.

FIG. 5 shows the plasmid profile of expression vector 5.

FIG. 6 shows the statistical graphs of the relative mRNA expression level (6A) and protein expression level (6B) of the RS1 gene in HEK293T cells after infection with AAV virus particles packaged with expression vectors 1-5.

FIG. 7 shows the retinal HE staining graph (7A) and retinal thickness statistical graph (7B) of each group of mice; in which, “ONL” refers to the outer nuclear layer of the retina, and “INL” refers to the inner nuclear layer of the retina.

FIG. 8 shows the optomotor response statistical graphs (8A and 8B) and visual acuity statistical graph (8C) of mice in each group under scotopic and photopic conditions, in which, in FIG. 8C, each column from left to right represents the control group, treatment model group, and non-treatment model group.

FIG. 9 shows the waveform (9A) and statistical graphs of amplitudes (9B-9E) of ERG (electroretinogram) of mice in each group.

### DETAILED DESCRIPTION

The principles and features of the present application are described below in conjunction with the accompanying drawings. The examples given are only for the purpose of explaining the present application and are not intended to limit the scope of the present application.

### 1. Construction of AAV-RS1 Recombinant Expression Vector

It was found in the research that using different RS1 expression vectors can produce different therapeutic effects on retinoschisis. In order to find the optimal expression vector, three generations of AAV recombinant expression vectors were designed using pAAV-MCS-ITR as the backbone. The first generation is expression vector 1, the second generation is expression vector 2, and the third generation is expression vectors 3-5.

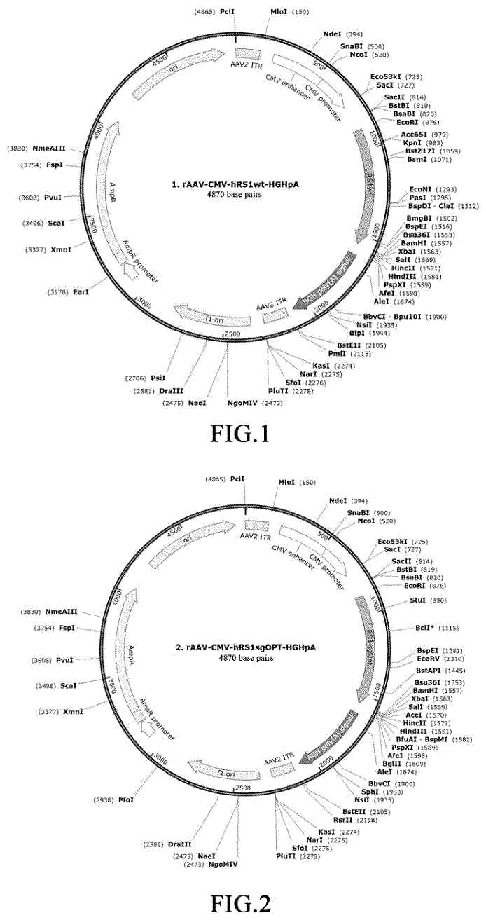

The structures of expression vectors 1 and 2 are shown in FIGS. 1 and 2, respectively, which are rAAV2/8-CMV-hRS1-HGHpA, in which CMV refers to the CMV promoter shown as SEQ ID NO: 1, HGHpA is human growth hormone polyadenylation, the sequence of which is shown as SEQ ID NO: 3. The difference lies in the use of different RS1 coding sequences, in which the hRS1wt used in expression vector 1 is the RS1 wild-type sequence, as shown in SEQ ID NO: 2, and the hRS1sgOPT used in expression vector 2 is the codon optimized RS1 gene, the sequence of which is shown as SEQ ID NO: 6.

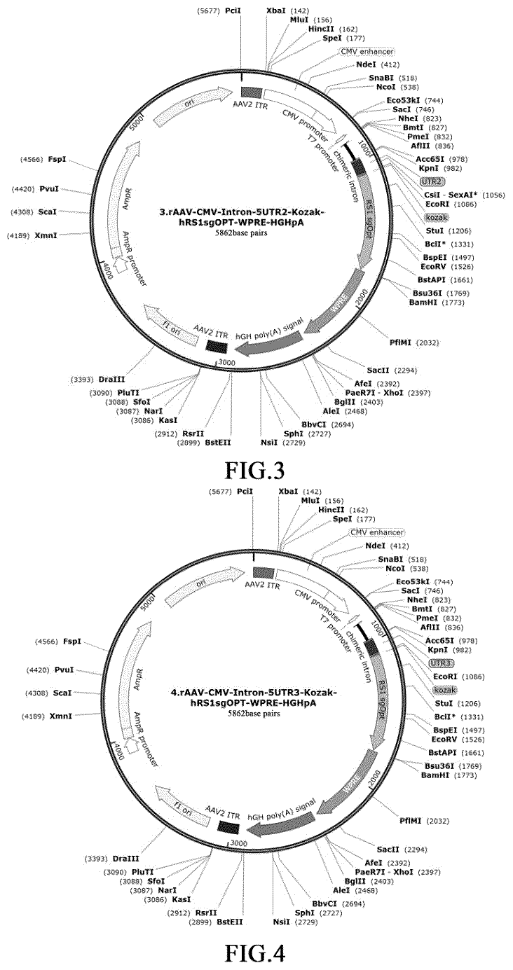

The structures of expression vectors 3-5 are shown in FIGS. 3-5, which are rAAV2/8-CMV-Intron-5′UTR-Kozak-hRS1sgOPT-WPRE-HGHpA, in which CMV refers to the CMV promoter, and intron is an intervening sequence in eukaryotic cell DNA, which is transcribed into precursor RNA, removed through splicing, and ultimately does not exist in mature RNA molecule. We used an artificially synthesized chimeric intron that enhances processing of mRNA and increases the expression level of downstream gene coding frame, as shown in SEQ ID NO: 4. In the eukaryotic expression system, Kozak sequence typically can enhance the efficiency of transcription and translation, as shown in SEQ ID NO: 5; HRS1sgOPT is a codon optimized RS1 gene; Woodchuck hepatitis virus posttranscriptional regulatory element (WPRE) is a DNA sequence, which produces a tertiary structure that enhances expression when transcribed, and can increase the expression level of an exogenous gene delivered by the viral vector. The sequence of WPRE is shown as SEQ ID NO: 7; HGHpA is a human growth hormone (hGH) polyadenylation (PolyA) that plays an important role in maintaining mRNA stability and avoiding its degradation during mRNA extracellular transport and translation.

Studies have shown that rational design of the 5′ untranslated region (5′UTR) can increase the expression level of exogenous proteins. The difference between expression vectors 3-5 lies in the use of different 5′UTRs. In the experiment, multiple 5′UTR sequences were designed and it was found that 5′UTR-2 (SEQ ID NO: 8) and 5′UTR-3 (SEQ ID NO: 9) have good effects. The 5′UTR of expression vector 3 is 5′UTR-2, the 5′UTR of expression vector 4 is 5′UTR-3, and the 5′UTR of expression vector 5 is 5′UTR-2 concatenated with 5′UTR-3, in which the two sequences are connected by a linker, namely 5′UTR-2-3 (SEQ ID NO: 10).

### 2. Preparation and Purification of AAV Virus

PEI transfection reagent was used to co-transfect three plasmids, namely, Rep and Cap protein expression plasmids of AAV8 (pAAV-RC2/8), a helper plasmid (pAd-helper), and an AAV core expression plasmid carrying an exogenous gene, into HEK293 cell to package and prepare AAV virus. After 96 hours of transfection, the cells and culture supernatant were harvested. After concentrating the virus crude solution through tangential filtration (TFF), the AAV virus was further purified using iodixanol density gradient ultracentrifugation, followed by desalination and concentration by centrifugation using a dialysis tube of Amicon Ultra 100K. The titer of purified virus was determined using fluorescence quantitative PCR (Q-PCR) method, the purity of the virus was detected by SDS-PAGE staining, and the endotoxin content was detected by tachypleus amebocyte lysate. Finally, the titer of AAV virus was adjusted to 2×10¹² vg/mL. The purified AAV viruses were subpackaged and stored in a freezer of −80° C.

3. Comparison of In Vitro Expression Efficiency of HEK293T Cells Infected with AAV Virus
1) Infection of HEK293T cells: the 6-well plates were divided into 5 experimental groups, and 5×10⁵ HEK293T cells were inoculated into each well. After 1 day of inoculation, the cell count was about 1×10⁶, and the cells were infected with 50 μl (1×10¹⁰ vg) of AAV viruses, i.e., expression vector 1 virus particles (group A), expression vector 2 virus particles (group B), expression vector 3 virus particles (group C), expression vector 4 virus particles (group D), and expression vector 5 virus particles (group E), respectively. 48 hours after infection, mRNA and protein were extracted from infected cells for detection and analysis.
2) Q-PCR detection of relative expression level of RS1 gene at mRNA level: Firstly, total RNA of cells was extracted using Trizol kit and reverse transcribed into cDNA template. Then, fluorescence quantitative PCR was used for detection. Q-PCR reaction system is as follows: 10 μl of SYBR Green II; 0.3 μl each of the target fragment primers; 0.4 μl of ROX; 20 μl of sterile water. The Q-PCR reaction procedure is: predenaturation at 95° C. for 5 minutes; and 40 cycles of 95° C. for 15 seconds, and 60° C. for 1 minute. The 2-ΔΔCt relative quantification method was used to analyze the differences in RS1 gene expression levels.
3) Western blot detection of the relative expression level of RS1 gene at the protein level: HEK293T cells from different groups were isolated, and washed once with PBS after the culture medium was removed. After adding lysis buffer for full lysis, the cells were centrifuged at 12000 g for 5 minutes and the supernatant was collected. The protein concentration was measured by BCA method, and then the sample volume of each group was calculated according to the total protein 30 μg. SDS-PAGE gel electrophoresis and Western blot detection were performed, and the gray scale quantitative analysis was performed by Image J software.

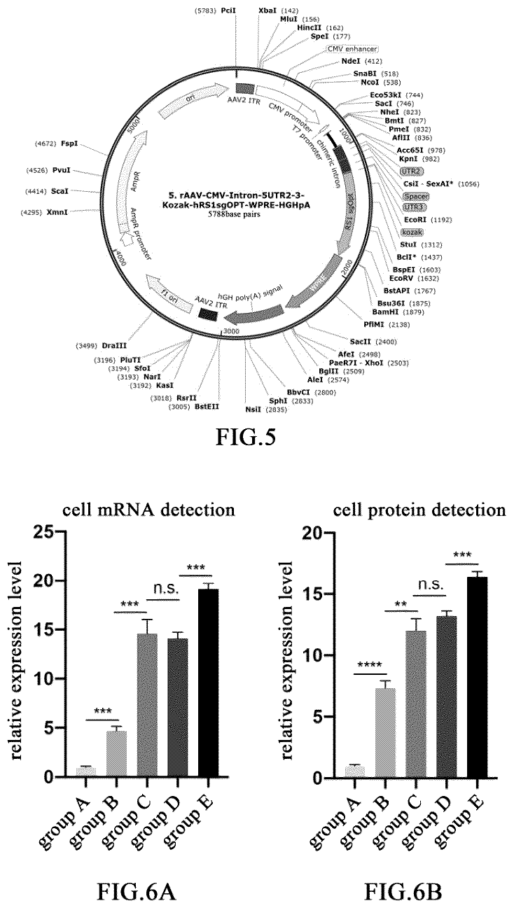

As shown in FIG. 6, both at the mRNA level (FIG. 6A) and protein level (FIG. 6B), the relative expression levels of the target protein are significantly increased in groups B, C, D, and E compared to group A, in which the relative expression levels of the target protein in group B increase by more than 7 times, groups C, D, and E increase by more than 12 times. From this, it can be seen that the expression level of the RS1 gene significantly is increased by codon optimization, and further boosted by the design of non-coding regulatory sequence. After optimization, the second-generation and third-generation vectors have much higher in vitro infection and expression efficiency, especially the expression level of group E (expression vector 5 formed by concatenating two 5′UTRs) is more than 16 times that of group A (expression vector 1). Therefore, expression vector 5 was chosen for the subsequent animal experiments.

### 4. Construction of RS1h KO in XLRS Model Mice

The XLRS model mouse RS1h KO was obtained by knocking out the first exon and a 1.6×10³ bp of partial the first intron of the homologous gene RS1h of the mouse RS1 using CRISPR/Cas9 technology. The brief process is as follows. Firstly, Cas9 mRNA and sgRNA targeting the target gene RS1h were prepared, and then are directly injected into 0.5-day-old C57BL/6J mouse fertilized eggs through microinjection technology. Finally, the fertilized eggs were transplanted into the fallopian tube of the pseudo pregnant mother to obtain F0 generation mice. Through PCR genotype identification and sequencing verification, F0 generation mice that meet the requirements were screened, and then were bred with C57BL/6J mice to obtain stable F1 generation positive mice. The tail of offspring mouse was cut for identification using PCR technology. The wild-type C57BL/6J mice used in the experiment were purchased from Beijing Vital River Laboratory Animal Technology Co., Ltd. All mice were fed and bred in an SPF barrier environment (22±2° C., 60±10% relative humidity, 12h/12h light dark cycle) at the animal experiment center of Medical Research Institute, Wuhan University, with arbitrary intake of food and water. All animal experiments and procedures have been approved by the Animal Ethics Committee of the Medical Research Institute, Wuhan University, and strictly follow the “The Guide for Care and Use of Laboratory Animals”.

### 5. Recovery of Retinal Structure and Visual Function in RS1h KO Mice by RS1 Expression Vector Virus Particles

The RS1 expression vector virus particles prepared from expression vector 5 were topically administered intraocularly, i.e. via subretinal or intravitreal injection. In the present application, 1 μl of recombinant viral preparation was directly injected into one eye of P22=3 RS1h-KO mouse through a vitreous cavity to express the human RS1 protein in the mouse retina, thereby achieving the restoration of retinal structure and visual function. Specific steps: After fully dilating the pupils of mice with compound tropicamide eye drops, the mice were generally anesthetized with 5% chloral hydrate. Under a dissecting microscope, a self-assembled microinjector and a 32 GB insulin NovoFine were used to insert the needle diagonally into the vitreous cavity 1 mm behind the cornea limbus, avoiding the iris and crystalline lens. 1 μl of dose was slowly injected and the needle was kept stopping for 2 minutes. After surgery, levofloxacin eye ointment was applied once a day for three consecutive days to reduce inflammation and prevent infection.

At 4 weeks after treatment, the recovery of the retinal structure and visual function of RS1h-KO mice was detected by HE staining, visual motor and ERG.

### 5.1 Retinal Structure

Detection of retinal structure by HE staining: The mouse was euthanized by cervical dislocation method after deep anesthesia, and the eyeball was removed, washed with PBS, and fixed with 4% paraformaldehyde for 24 hours. After conventional gradient ethanol dehydration, the eyeball was soaked in xylene at 60° C. for 10-15 minutes and paraffin-embed, and sliced along the sagittal axis parallel to the optic nerve with a thickness of 4 μm. After HE staining and sealing the slice, observation was performed under an optical microscope. The thicknesses of the inner nuclear layer, outer nuclear layer and the entire retina on both sides 500 μm away from the optic nerve were measured by using Image J software.

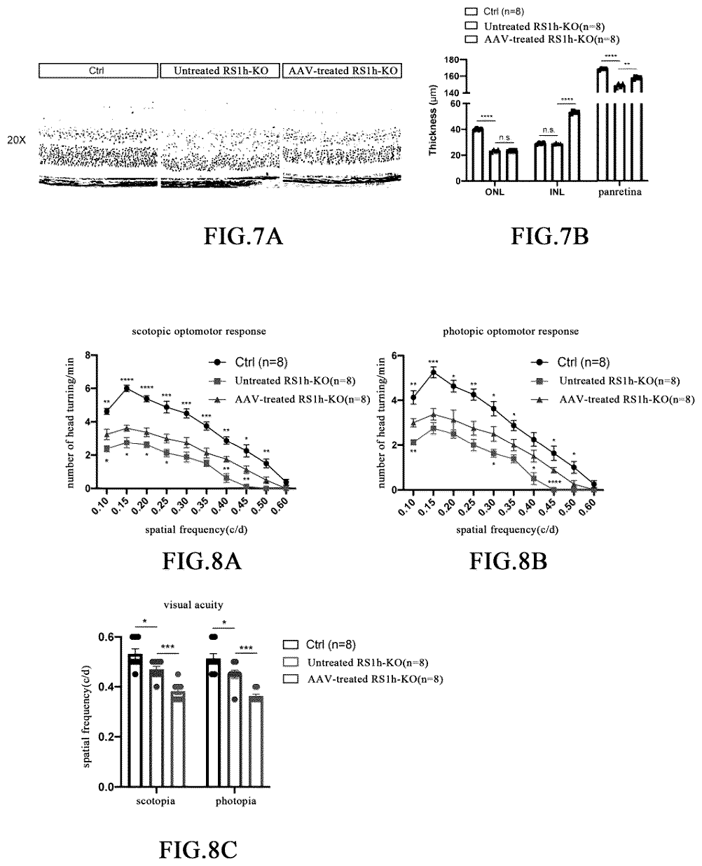

The results are shown in FIG. 7A. After 4 weeks of treatment, the structural arrangement of each layer in the treated eyes of RS1h KO mice returns to near normal, and the split sac cavity almost completely disappears, while the retina structure of the untreated fellow eye is slightly disordered, and the split sac cavity still exists. FIG. 7B indicates that the thickness of the inner nuclear layer (INL) of the retina in treated eye of the RS1h-KO mouse significantly decreases, approaching normal levels.

### 5.2 Optomotor Response

The optomotor response was detected by the following method. After adapting for 3-4 hours in the dark (dark adaptation condition) or 10 minutes under 400 lux of light stimulation (light adaptation condition), the mouse was placed on a platform (with a diameter of 8.5 cm, a height of 17.5 cm from the bottom mirror surface, located at the center of the mirror surface) for free movement. There were two mirrors above and below the platform (with a 24 cm diameter of circle hollowed out in the center of the upper mirror surface), and a camera was placed on the top mirror for recording. The platform was surrounded by four LCD displays, which can display moving gratings of different frequencies (the spatial frequency of the gratings increases from 0.1 to 0.6 cyc/deg: 0.1, 0.15, 0.2, 0.25, 0.3, 0.35, 0.4, 0.45, 0.5, 0.6 cyc/deg; the grating at each frequency rotated alternately in the clockwise and counterclockwise direction for 30 seconds in each direction, with a pause of 10 seconds therebetween). When the mice saw the movement of the grating around them, they will make a tracking behavior of turning their heads. The visual acuity was quantified by analyzing the head turning of mice under moving gratings of different frequencies.

As shown in FIG. 8, the number of head turning in the optomotor response of the treated eye of RS1h-KO mouse under the scotopic and photopic conditions is greater than that of the fellow eye, and some differences are statistically significant (FIGS. 8A and 8B). At the same time, the vision of the treated eye shows significant improvement under both the scotopic and photopic conditions (FIG. 8C).

### 5.3 ERG Reaction

The ERG (electroretinogram) response was detected by the following method. After overnight in the dark environment (scotopic condition) or continuous stimulation under system background light for 10 minutes (photopic condition), the compound tropicamide eye drops were dropped on the surfaces of both eyes of the mouse for mydriasis. Subsequently, the mouse was anesthetized by intraperitoneal injection of 5% chloral hydrate at a dose of 0.01 mL/g. After anesthesia, Oxybuprocaine Hydrochloride eye drops were applied topically for ocular surface anesthesia. After entering into the anesthesia state, the mouse was connected with the electrodes, in which recording electrode slightly contacted with the top center of the mouse cornea vertically, and the reference electrode and ground electrode were inserted subcutaneously between the two ears and inserted into the tail of the mouse, respectively. All operations were performed under dark red light to ensure the dark adaptation state of the mouse. Measurement was performed using the RetiMINER 4.0 ocular electrophysiological visual system, in which the light intensity stimulation was increased successively and the comprehensive potential response of retina was recorded. The a-wave amplitude wave is the potential difference from baseline to the a-wave valley, and b-wave amplitude is the potential difference from a-wave valley to b-wave peak.

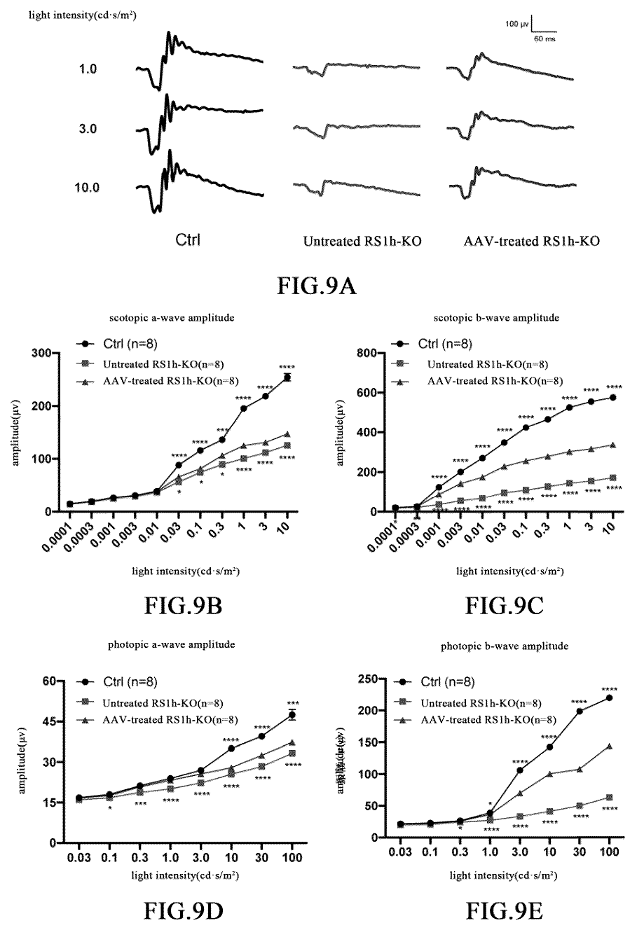

As shown in FIG. 9, the amplitudes of a and b waves of the treated eye of RS1h-KO mouse are higher than those of the fellow eye under all light intensities in ERG detection, which is statistically significant under almost all medium to high light intensity, even some low light intensity. Overall, 4 weeks after treatment by intravitreal injection of the virus, the amplitudes of a and b waves in RS1h KO mouse under the highest light intensity can be roughly recovered to half the level of normal mouse.

Similar in vivo experimental results can also be obtained using expression vectors 3 and 4. Although expression vector 2 is more effective than expression vector 1, it is not as effective as the third generation of expression vectors (expression vectors 3-5).

Although the embodiments of the present application have been shown and described above, it can be understood that the above embodiments are exemplary and should not be construed as limitation of the present application. Those of ordinary skill in the art may make changes, modifications, substitutions, and variations to the above embodiments within the scope of the present application.

The correspondence between the sequence number and the sequence name in the present application is shown in Table 1.

TABLE 1
Correspondence between the sequence number and
the sequence name in the present application

| Sequence number | Sequence name |
|---|---|
| SEQ ID NO: 1 | CMV promoter |
| SEQ ID NO: 2 | RS1 wild type |
| SEQ ID NO: 3 | HGHpA sequence |
| SEQ ID NO: 4 | Intron sequence |
| SEQ ID NO: 5 | Kozak sequence |
| SEQ ID NO: 6 | codon optimized RS1gene |
| SEQ ID NO: 7 | WPRE sequence |
| SEQ ID NO: 8 | 5′UTR-2 |
| SEQ ID NO: 9 | 5′UTR-3 |
| SEQ ID NO: 10 | 5′UTR-2-3 |

For the specific sequences of SEQ ID NO: 1 to SEQ ID NO: 10, see the sequence list.

1. Use of RS1 gene in preparing a therapeutic agent for X-linked retinoschisis.

2. The use of claim 1, wherein a sequence of the RS1 gene is shown as SEQ ID NO: 6.

3. A therapeutic agent for X-linked retinoschisis, comprising an RS1 gene expression vector, wherein the RS1 gene expression vector comprises an RS1 gene expression cassette.

4. The therapeutic agent of claim 3, wherein a sequence of the RS1 gene in the RS1 gene expression cassette is a codon optimized sequence, which is shown as SEQ ID NO: 6.

5. The therapeutic agent of claim 3, wherein a promoter that controls the expression of the RS1 gene is a CMV promoter.

6. The therapeutic agent of claim 4, wherein the RS1 gene expression cassette further comprises a non-coding regulatory sequence, which comprises one or more of an Intron sequence, a Kozak sequence, a 5′UTR sequence, a WPRE sequence, and a HGHpA sequence.

7. The therapeutic agent of claim 6, wherein the RS1 gene expression cassette is CMV-Intron-5′UTR-Kozak-hRS1-WPRE-HGHpA.

8. The therapeutic agent of claim 6, wherein the Intron sequence is shown as SEQ ID NO: 4, the Kozak sequence is shown as SEQ ID NO: 5, the WPRE sequence is shown as SEQ ID NO: 7, the HGHpA sequence is shown as SEQ ID NO: 3, and the 5′UTR sequence is selected from the group consisting of SEQ ID NO: 8 and SEQ ID NO: 9, or is composed of SEQ ID NO: 8 and SEQ ID NO: 9 connected in series.

9. The therapeutic agent of claim 8, wherein the 5′UTR sequence is composed of SEQ ID NO: 8 and SEQ ID NO: 9 connected in series, and is shown as SEQ ID NO: 10.

10. The therapeutic agent of claim 3, wherein the expression vector is an adeno-associated virus expression vector, which is packaged in an adeno-associated virus particle.

11. The therapeutic agent of claim 10, wherein the adeno-associated virus is AAV2/8.

12. Use of the RS1 gene of claim 1, or a therapeutic agent for X-linked retinoschisis, comprising an RS1 gene expression vector, wherein the RS1 gene expression vector comprises an RS1 gene expression cassette for treating X-linked retinoschisis.

13. Use of the therapeutic agent of claim 3 in the preparation of a medicament for treating X-linked retinosplits.

14. A method for treating X-linked retinoschisis, comprising administering the therapeutic agent of claim 3 to a subject in need thereof.


SEQUENCE LISTING (10 sequences)

SEQ ID NO: 1 - DNA, 584 bp, synthetic construct
  source 1..584: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 2 - DNA, 675 bp, Homo sapiens
  source 1..675: mol_type = other DNA
atgtcacgcaagatagaaggctttttgttattacttctctttggctatgaagccacattgggattatcgtctaccgaggatgaaggcgaggacccctggtaccaaaaagcatgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcagggatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctga

SEQ ID NO: 3 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 4 - DNA, 133 bp, unidentified
  source 1..133: mol_type = other DNA
gtaagtatcaaggttacaagacaggtttaaggaggccaatagaaactggccttgtcgagacagagaagattcttgcgtttctgatagccacctattggtcttactgacatccactttgcctttctctccacag

SEQ ID NO: 5 - skipped

SEQ ID NO: 6 - DNA, 675 bp, synthetic construct
  source 1..675: mol_type = other DNA
atgagcagaaagatcgagggattcctgctgctgctgctgtttggctatgaggccaccctgggcctgtctagtaccgaggatgagggagaagatccttggtatcagaaggcctgtaaatgtgattgtcagggcggccctaatgccctgtggagcgccggagctaccagcctggattgtattcctgagtgtccttaccacaagcctctgggatttgagagcggagaagtgacccctgatcagatcacctgtagcaatcctgagcagtatgtgggctggtactcttcttggacagctaataaggccagactgaatagccagggatttggctgcgcttggctgagcaagtttcaggactctagccagtggctgcagatcgatctgaaggaaatcaaagtgatttccggaatcttgacccagggaagatgtgatatcgatgagtggatgaccaagtacagcgtgcagtatagaacagatgagagactgaattggatctattataaggaccagacaggcaataatagagtgttttacggcaattctgatagaacaagcaccgtgcagaatctgctgagaccacctattatttctaggtttatcagactgattcctctgggctggcacgtgagaatcgccatcagaatggagctgctggagtgcgtgagcaagtgtgcctga

SEQ ID NO: 7 - DNA, 600 bp, synthetic construct
  source 1..600: mol_type = other DNA
gcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 8 - DNA, 100 bp, synthetic construct
  source 1..100: mol_type = other DNA
cactcgcgctgccatcactcttccggcgtcttcgccgccatcctcgccgcgactcgcttctttcggttctaccaggtagagtccggcgccatcctccacc

SEQ ID NO: 9 - DNA, 100 bp, synthetic construct
  source 1..100: mol_type = other DNA
cttgtctcgctccggggaacgctccgaaactcccggccgccgccaccggcgtctgttctgttacacaaggcaagaaaagccgctgccgcactccgagtgt

SEQ ID NO: 10 - DNA, 206 bp, synthetic construct
  source 1..206: mol_type = other DNA
cactcgcgctgccatcactcttccggcgtcttcgccgccatcctcgccgcgactcgcttctttcggttctaccaggtagagtccggcgccatcctccacccaacaacttgtctcgctccggggaacgctccgaaactcccggccgccgccaccggcgtctgttctgttacacaaggcaagaaaagccgctgccgcactccgagtgt